(19) United States
(12) Patent Application Publication
(10) Pub. No.: US 2019/0057771 A1
(43) Pub. Date: Feb. 21, 2019

(54) SIMPLIFIED LAUNCHING OF ELECTRONIC MESSAGES IN CENTER FOR TREATING PATIENT

(71) Applicant: Physio-Control, Inc., Redmond, WA (US)

(72) Inventors: G. Cees Verkerk, Seattle, WA (US); Dana S. Lewis, Woodinville, WA (US); Randy L. Merry, Woodinville, WA (US)

(21) Appl. No.: 16/105,919

(22) Filed: Aug. 20, 2018

Related U.S. Application Data
(63) Continuation of application No. 14/703,382, filed on May 4, 2015, now abandoned.
Application No. 14/703,382 is a continuation of application No. 12/976,919, filed on Dec. 22, 2010, now abandoned.
(60) Provisional application No. 61/291,999, filed on Jan. 4, 2010.

(51) Int. Cl. G16H 40/63; G06Q 10/10; G06F 19/00; G06Q 50/22; G16H 10/60

(52) U.S. Cl. CPC G16H 40/63; G06Q 50/24; G06Q 10/10; G06F 19/00; G06F 19/3481; G16H 10/60; G06Q 50/22

(57) ABSTRACT

Devices, systems, software and methods facilitate launching protocol in a treatment center for an incoming patient. When a patient characterization is input, one or more message templates become prepared at least in part for different functions, departments and people of the treatment center.

### CROSS-REFERENCE TO RELATED PATENT APPLICATIONS

This patent application is a continuation of U.S. patent application Ser. No. 14/703,382, Filed May 4, 2015, which is a continuation of U.S. patent application Ser. No. 12/976,919, entitled “SIMPLIFIED LAUNCHING OF ELECTRONIC MESSAGES IN CENTER FOR TREATING PATIENT,” filed Dec. 22, 2010, which claims priority from U.S. Provisional Patent Application Ser. No. 61/291,999, filed on Jan. 4, 2010, both of which are hereby incorporated by reference herein.

### FIELD

This invention generally relates to electronic communications techniques in centers for treating patients, such as hospitals, emergency care centers, and the like.

### BACKGROUND

When a patient is being brought into a treatment center, they are often characterized according to their ailment. For example, a patient can be characterized as a “trauma patient”, or a “STEMI patient”, or something else. STEMI stands for ST-segment Elevated Myocardial Infarction, which is a heart-related ailment. These characterizations are usually made by an authorized individual, such as a physician on staff, who has reviewed & diagnosed medical data associated with the patient. The characterization may be made by someone in direct proximity to the patient, or someone who is consulted remotely.

In situations where a patient is being brought in an emergency, the treatment center has to be notified quickly, because time is of the essence. More particularly, even for a single patient, multiple departments may have to be notified, which poses coordination problems. Examples 30 are now given.

FIG. 1 is a conceptual diagram to illustrate the coordination problem in the prior art. Within a single treatment center 140, a nurse might have to coordinate by telephone with persons in multiple departments, and that can be for a single incoming patient with a heart problem. The coordination is so that these persons perform their tasks, like start specific procedures, order specific tests, etc.

FIG. 2 is a diagram showing electronic communication components of a modern treatment center 240. Instead of using telephones, a computer system 220 is employed, along with a communication network, such as an intranet. Computer system 220 is networked to a variety of network destinations within treatment center 240. Three destinations are shown, namely destination A 270, destination B 280, and destination C 290, although more are possible and more are usually implemented. These destinations are for the persons who treat patients, and/or operate the type of facilities, as shown in FIG. 1.

More particularly, when a new patient 200 is being brought in to treatment center 240, a characterization 212 is usually provided. Then someone at center 240, such as a nurse, has to notify the individual departments based on this characterization. That someone operates a regular interface 230 of computer system 220, according to operation 242. As such, they generate, serially and from the beginning, individual messages for various network destinations. These can be email messages, pager messages, text-to-voice messages, etc. In the example of FIG. 2, two such messages MSG1 264, MSG2 266 are shown, for respective network destinations 270, 290. Which individual messages are to be generated for which destinations is, of course, a matter of which medical protocol the sender has to look up and follow for the learned patient characterization 212. Typically, a medical director of a treatment center decides on the protocol that is to be looked up by the creator of the messages.

The improvement of FIG. 2 has not fully solved the problem of FIG. 1. In operation 242, crafting messages MSG1 264, MSG2 266, and routing them to their appropriate destinations, can still be laborious, time consuming, and prone to error. In emergency situations, errors can cost lives.

### BRIEF DESCRIPTION OF THE DRAWINGS

FIG. 1 is a conceptual diagram illustrating the prior art problem of coordinating care for a patient incoming in a treatment center.

FIG. 2 is a diagram showing electronic communication components of a modern treatment center, but in which the coordination problem of the prior art has simply taken a different form.

FIG. 3 shows the treatment center of FIG. 2, but which has been retrofitted with a launch utilities and interface for a computer system according to embodiments.

FIG. 4 is a conceptual diagram illustrating how the launch utilities of FIG. 3 solve the coordination problem of FIG. 1 and FIG. 2.

FIG. 5 is a block diagram showing a detailed embodiment for implementing the launch utilities of FIG. 3.

FIG. 6 is a flowchart for illustrating methods for simplified launching of electronic messages in a patient treatment center according to embodiments.

FIG. 7 is a composite diagram for illustrating methods according to embodiments for generating message templates for one or more operations of the flowchart of FIG. 6.

### DETAILED DESCRIPTION

As has been mentioned, the present description is about devices, systems, software and methods for automatically launching electronic messages in a treatment center. It will be appreciated that the embodiments described herein may be implemented, for example, via computer-executable instructions or code or a computer implemented process, such as launch utilities 330, stored on a computer-readable storage medium and executed by a computing device. Generally, a program involves routines, objects, components, or data structures, and the like that perform particular tasks or implement particular abstract data types. As used herein, the term “program” may connote a single program or multiple programs acting in concert, and may be used to denote applications, services, or any other type or class of program. Likewise, the terms “computer” and “computing device” as used herein include any device that electronically executes one or more programs, including, but not limited to, personal computers, servers, laptop computers, hand-held devices, cellular phones, microprocessor-based programmable consumer electronics and other computer image processing devices. Embodiments are now described in more detail.

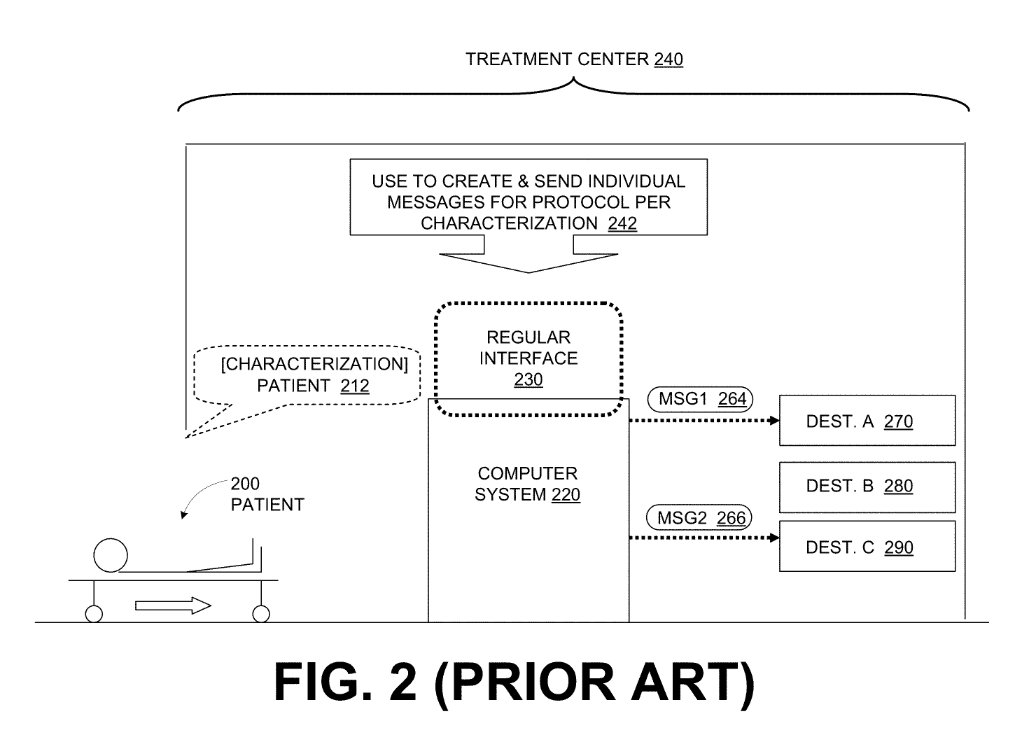

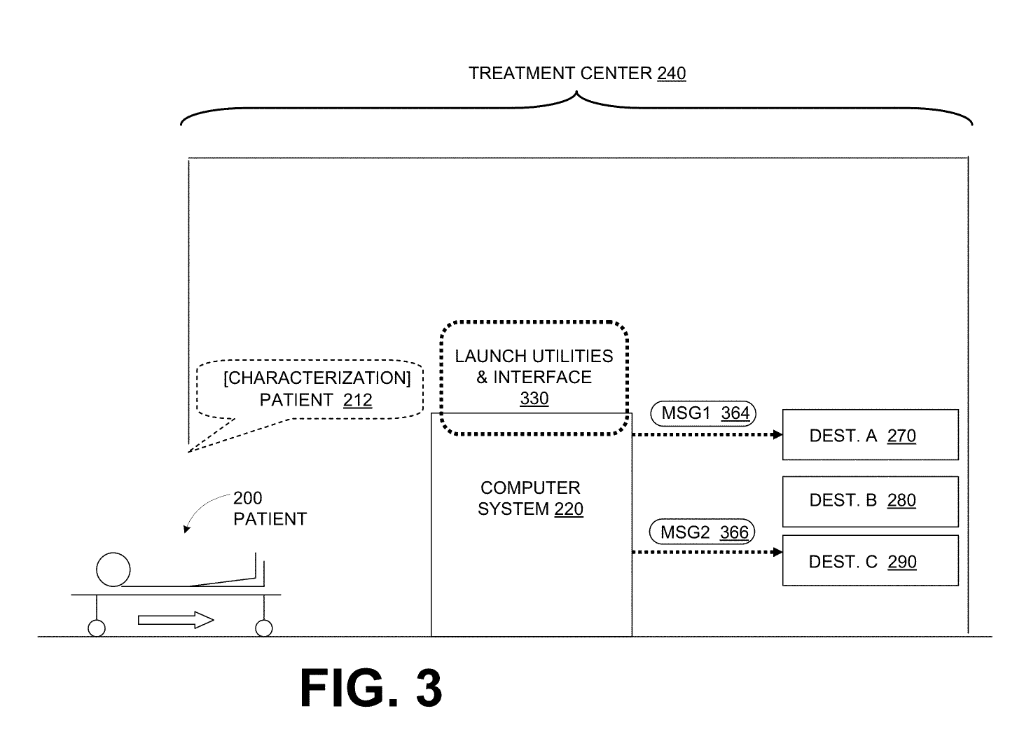

FIG. 3 shows treatment center 240 that was already described with reference to FIG. 2.

But there can be differences and extensions. For example, computer system 220 can be connected to the internet. As such, network destinations can even be outside treatment center 240. In addition, for computer system 220, a launch utilities and interface 330 are provided, made according to embodiments. It will be understood, of course, that this is by example and not by limitation. For example, the invention may be implemented over a single computer, or multiple computers, and so on.

Launch utilities and interface 330 can help launch messages such as MSG1 364, MSG2 366, even for the same network destinations as in the prior art. The messages of launch utilities and interface 330 can be email messages, text messages, pager messages, application-to-application messages, and so on. But MSG1 364, MSG2 366, can be even identical to messages MSG1 264, MSG2 266. Notwithstanding that, it is the internal workings of launch utilities and interface 330 that make it substantially easier on the user to generate these messages. This is illustrated below.

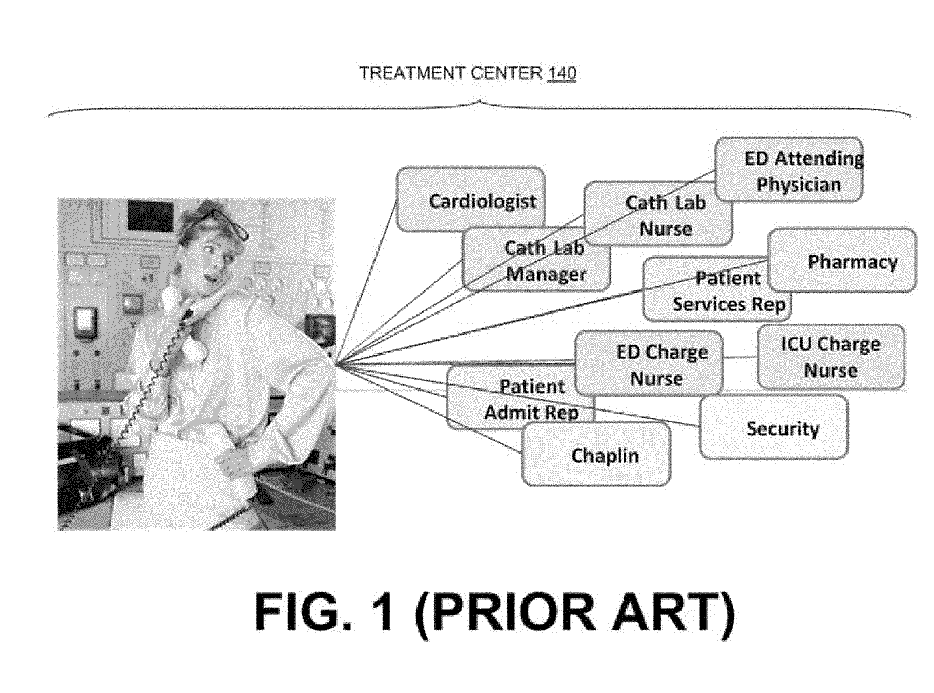

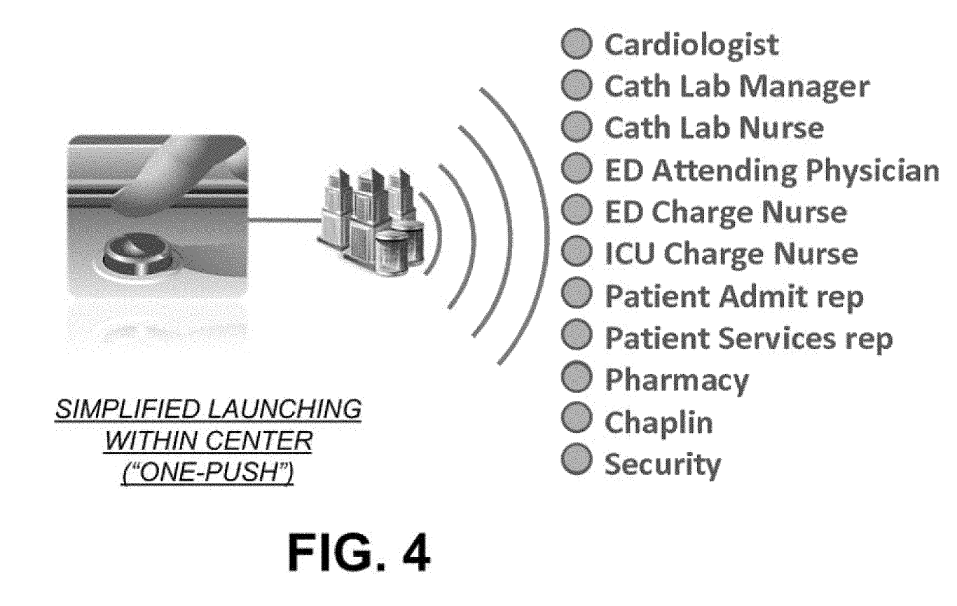

FIG. 4 is a conceptual diagram illustrating how the launch utilities of FIG. 3 solve the coordination problem of FIG. 1 and FIG. 2. It is as if the user pushes a single button one time, and this launches the right messages for the right facilities and persons become notified. Of course, people understand that there is no physical button that is being pushed, although there could be. Later description will show that the “button” is typically something that can be presented by a user interface, and so on. Plus, while at least two messages need to be launched with a single “push”, not all of them need to be so launched.

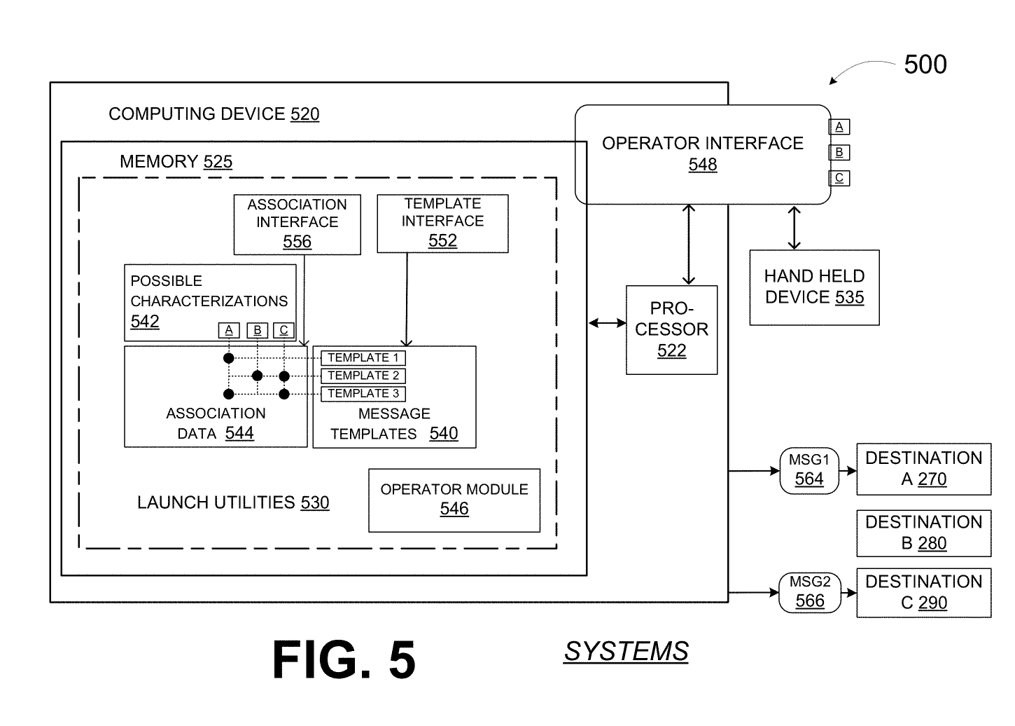

FIG. 5 is a block diagram showing a detailed embodiment for implementing the launch utilities of FIG. 3. A system 500 can be used to automatically launch electronic messages in center 240. System 500 can be implemented using a computing device 520, which can be like computer system 220.

Computing device 520 includes a processor 522, and a memory 525 in communication with processor 522. Memory 525 can be implemented by one or more chips. Launch utilities 530 may be provided on memory 525, or otherwise be provided applications loaded on distributed computer systems, hand held devices such as cellular phones and personal data assistants, and are now described in more detail.

Possible characterizations of ailments 542 may reside in memory 525, for example as suitable encodings. Only characterizations A, B, C are shown, but more are possible. These could be, for example, “Trauma”, “STEMI”, and another that is designated by the medical director of the center, as are the capabilities of the center.

Message templates 540 may also reside in memory 525, for example as suitable encodings. Three templates TEMPLATE 1, TEMPLATE 2, TEMPLATE 3 are shown, but more are possible. Message templates 540 can be specific for the intended ones of possible destinations 270, 280, 290 of the center.

Association data 544 may further reside in memory 525, for example as suitable encodings. Association data 544 may associate at least some of the possible characterizations 542 with at least some of message templates 540. The association is indicated conceptually in FIG. 5 as a matrix, with circular dots where an association is valid. Thus, in the example of FIG. 5, characterization A is associated with templates TEMPLATE 1 and TEMPLATE 3, characterization B is associated with template TEMPLATE 2, and characterization C is associated with templates TEMPLATE 2 and TEMPLATE 3.

Utilities 530 may further include an operator module 546, which can reside as executable instructions in memory 525. Operator module 546 may input a learned characterization of an ailment of an incoming patient. Such inputting can operate as a launch command as it can launch messages, as will be described later in this document. The learned characterization can be input in any number of ways. In the preferred embodiment, inputting is via an operator interface 525, which can be provided as an optional part of utilities 530. More particularly, an incoming message can be received by operator module 546 via operator interface 548, which encodes the learned characterization.

When the learned characterization according to the patient's ailment is input, it can be compared with the stored possible characterizations 542. If one of possible characterizations 542 is matched with the learned characterization, then one or more of message templates 540 associated with the matched characterization can be looked up automatically, from association data 544. Then one or more message templates can be caused to be prepared automatically for transmission, using the looked up message templates. The transmission can be for one or more network destinations, such as destinations 270, 280, 290 of center 240. These message templates can be similar or different, and are generally intended to prepare persons or facilities at these destinations to treat the patient according to the ailment.

Operator interface 548 can be implemented as a graphical user interface, and is now described in more detail. In preferred embodiments, operator interface 548 includes options that the user can select as the characterization to be learned, and which correspond to stored possible characterizations 542. Thus, in the example of FIG. 5, operator interface 548 includes options A, B, C, which are further shown conceptually as pushbuttons. These interface options A, B, C correspond one-for-one with the possible characterizations 542, so that the above mentioned matching can take place. Indeed, when one of the interface options is selected by the user as the characterization to be learned, the corresponding one of the stored possible characterizations 542 becomes matched with the learned characterization as per the above.

Operator interface 548 can further include a screen. In some embodiments, one of the message templates is further caused to be displayed on the screen as part of being prepared. Moreover, the operator interface can enable the user to edit one of the message templates before sending it. In preferred embodiments, one or more of the message templates further includes a “To:” field that is already populated with a network address of one of the network destinations.

In some embodiments, operator interface 548 is operated on a hand held device 535, through which the patient characterization can be learned. In some embodiments, the hand held device provides the patient characterization through a wireless communication.

The message templates can be prepared according to rules that are preset in utilities 530, and can optionally be edited. In fact, possible characterizations 542, association data 544, and message templates 540, and other aspects of the message templates, can be considered part of a broader rules module. The rules module itself, or different components of it, can be editable by the user as background work, and preferably not during an emergency! Such background editing can be via additional interfaces, which can be implemented in any number of ways. In some embodiments, such additional interfaces can be implemented as extensions of operator interface 548, intended mainly for the local user. In others, such additional interfaces can be implemented remotely, for example by a system planner. In the example of FIG. 5, an optional template interface 552 can help edit one or more of templates 540, before it is looked up. Moreover, an optional association interface 556 can help adjust which of possible characterizations 542 are associated with which of message templates 540.

In some embodiments, different message templates are associated with rules which may depend on specific conditions. For example, rules can be applied without involving an operator, or different launching options can be presented to an operator according to the specific condition.

One example condition is time of day. So, if a learned characterization is input at a first time of day, a first set of message templates can be caused to be prepared, but a different set can be caused to be prepared at different time of day, even if the same learned characterization is input. For example, depending on whether the time is a) between 8 am-8 pm, or b) between 8 pm-8 am, different messages can be sent, or to different email addresses. The time can be consulted at the time of launch without involving the operator, or different launching options can be presented for her to choose, etc. Moreover, background editing can permit a planner to program the first and second times of day, and the first and second sets of messages.

Another example condition can be based on availability of people and facilities, e.g. checking upon schedules, etc. In some embodiments, an availability of at least one of the destinations is checked, and at least one of the drafted messages is caused to be prepared responsive to the availability. Checking can be by calendar functions, paging functions, etc. If the checked destination is shown as unavailable, another destination is used, and soon.

The above description was about preparing the message templates for transmitting. In some embodiments, one or more of the prepared message templates are further caused to be actually transmitted automatically, as part of the same sequence. In the example of FIG. 5, these are shown as MSG1 564, MSG2 566, being transmitted to respective network destinations 270, 290.

In some embodiments, transmission is not automatic. In some embodiments, the above mentioned background editing permits a planner to select between a) causing one or more of the prepared message templates to be transmitted automatically, and b) requiring a user to take a separate action so that the first prepared draft message can be transmitted. And, in other embodiments, such a separate action from the user is always required. The separate action can be for the user to click “Send”, or something equivalent. When a “Send” input is received from the user, one or more of the prepared message templates can be caused to be transmitted.

The invention can be further useful in conveying the appropriate patient data to the appropriate destinations. For example, an element of patient data can be learned about the patient. The patient data elements can be personal patient data, such as the gender, age or apparent age, along with more elaborate data such as ECG data. They can be received via a communications network such as the internet, or an application of it. They can be looked up from a record of the patient. Or they can be originated live from a medical device that is monitoring the patient, and the communications network can be coordinated to operate with the medical device. An example of such a communications network is the LIFENET® System by Physio-Control. In some embodiments, launch utilities and interface 330 are provided can be advantageously provided in conjunction with such a communications network.

Once learned, the patient data elements can be imparted in the one of the message templates, as appropriate. Imparting can be automatic, or not. In some embodiments, the above mentioned background editing permits a planner to select between a) the imparting being automated, and b) requiring a user to take a separate action to perform the imparting.

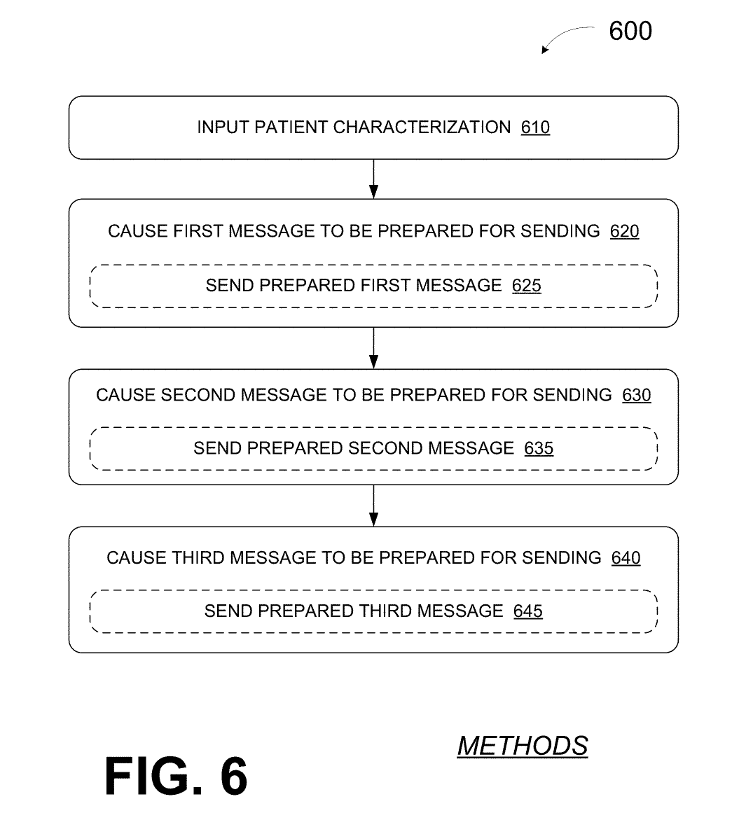

Messages 364, 366 can be logged and archived, so that historical information can be traced. Moreover, the invention can be further useful in monitoring response times in treatment centers. For example, after a first one of the prepared message templates is indeed caused to be transmitted to its network destinations, the system can wait for an acknowledgement to be received from a human operator at that network destination. The acknowledgement can be about a readiness to treat the patient by the person or facility of that destination. Statistics can then be computed and reported, such as a readiness time difference from when the prepared first draft message was indeed transmitted, to when the acknowledgement was received. FIG. 6 shows a flowchart 600 for describing methods according to embodiments. The method of flowchart 600 may also be practiced by the above mentioned embodiments. It will be recognized that many of the individual operations of flowchart 600 can be implemented as described above.

According to an operation 610, a learned patient characterization is input. According to a next operation 620, a first draft message is caused to be prepared for sending or transmitting. According to an optional next operation 625, the first draft message is actually transmitted.

According to a next operation 630, a second draft message is caused to be prepared for sending or transmitting. According to an optional next operation 635, the second draft message is actually transmitted.

According to a next operation 640, a third draft message is caused to be prepared for sending or transmitting. According to an optional next operation 645, the third draft message is actually transmitted.

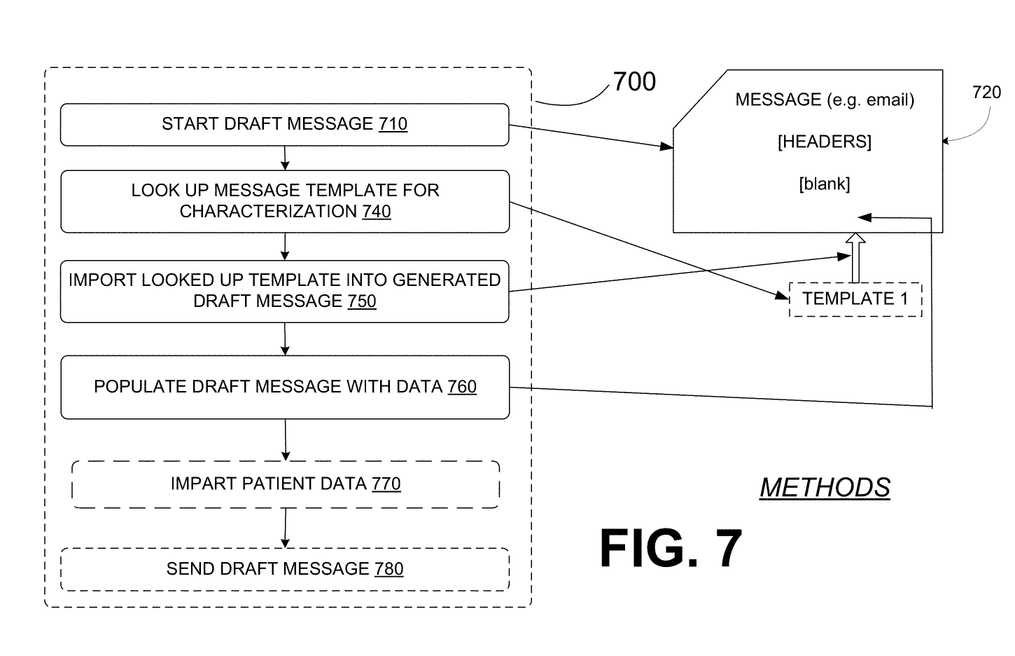

FIG. 7 is a composite diagram for illustrating methods according to embodiments for generating message templates for operations 620, 630, or 640 of the flowchart of FIG. 6. FIG. 7 includes a flowchart 700. It will be recognized that many of the individual operations of flowchart 700 can be implemented as described above.

According to an operation 710, a draft message is started. The message can be an email, a text message, and so on. A sample started message 720 is shown as an email, having one or more of header spaces, blank sections to store data, and to receive a message template as at least a portion of the message body of the draft email.

Additional background operation (not shown), rules can be consulted that may have been edited as a preparatory background operation. The rules can affect any and all operations of flowchart 700.

According to a next operation 740, a message template may be looked up that is associated with the characterization input. As per the above, looking up may be according to association data, and prevalent rules.

According to another operation 750, the looked up template may be imported into the started message.

According to another operation 760, the started message may be populated with data looked up from the rules.

According to an optional next operation 770, patient data may be imparted in the message.

According to an optional next operation 780, the message is sent.

In this description, numerous details have been set forth in order to provide a thorough understanding. In other instances, well-known features have not been described in detail in order to not obscure unnecessarily the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems.

The following claims define certain combinations and subcombinations of elements, features, steps, and/or functions, which are regarded as novel and non-obvious. Additional claims for other combinations and subcombinations may be presented in this or a related document.

1. A system for automatically launching electronic messages for treating patients in a center having at least two distinct network destinations, each destination associated with persons or facilities to treat the patients, the system comprising:
a memory configured to store:
a plurality of possible characterizations of ailments,
a plurality of message templates, and
association data that associates at least some of the possible characterizations with at least some of the message templates;
an operator module configured to receive a learned characterization of an ailment of an incoming patient; and
one or more processors configured to:
generate a first and a second draft message,
receive the learned characterization and identify at least one of a medical provider or a facility associated with treating the patient for the learned characterization,
determine whether the received characterization matches one of the possible characterizations,
if the received characterization matches one of the possible characterizations, transmit an inquiry to one or more of at least one of the medical providers or the facility to confirm availability, the inquiry including a request to send a response to confirm availability;
automatically select at least two different message templates from the plurality of message templates associated with the learned characterization that matches one of the possible characterizations,
apply one of the at least two different message templates to the first draft message to generate a first message and apply the other of the at least two different message templates to the second draft message to generate a second message, both the first message and the second message including the inquiry to the at least one of the medical provider or the facility,
cause the system to automatically prepare for transmission the first and second messages to at least two distinct network destinations based on the at least two different message templates,
select between causing a first message to be transmitted automatically, based on the at least one of the two different message templates, or requiring a user to perform a separate action through the operator module, based on the at least one of the two different message templates, to allow the first message to be transmitted, and
receive the response to confirm availability from the at least two distinct network destinations.

2. The system of claim 1, in which the operator module includes an operator interface, and the learned characterization is received from an incoming message received via the operator interface.

3. The system of claim 2, in which
the operator interface further comprises a plurality of selectable options, each corresponding to one of the possible characterizations, and
when one of the options is selected as the learned characterization, the corresponding one of the possible characterizations becomes matched with the learned characterization.

4. The system of claim 2, in which
the operator interface includes a screen, and
one of the first message or the second message is further caused to be displayed on the screen as part of being prepared for transmission.

5. The system of claim 1, in which the operator module enables a user to edit one of the message templates when the processor selects requiring a user to perform the separate action through the operator module.

6. The system of claim 1, in which one of the message templates further includes a “To:” field that is pre-populated with a network address of one of the network destinations.

7. The system of claim 1, in which the operator module operates on a hand held device.

8. The system of claim 7, in which the hand held device is configured to provide the learned characterization to the one or more processors through a wireless communication.

9. The system of claim 1, the operator module further comprising a template interface configured to enable a user to edit one of the message templates when the processor selects requiring a user to take the separate action through the operator module.

10. The system of claim 1, further comprising an association interface configured to enable adjusting which of the possible characterizations are associated with which of the message templates.

11. The system of claim 1, in which the processor is further configured to cause the first message and second message to be transmitted automatically.

12. The system of claim 1, in which the processor is further configured to select a first set of at least two different message templates when the learned characterization is input at a first time of day and to select a second set of at least two different message templates when the learned characterization is input at a second time of day.

13. The system of claim 1, in which the processor is further configured to receive an availability of at least one of the network destinations and if the destination is unavailable select another network destination.

14. The system of claim 1, in which at least one of the at least two different message templates includes a learned patient data.

15. The system of claim 14, in which the learned patient data is automatically imparted in the other one of the message templates.

16. The system of claim 14, in which the processor is further configured to look up from a record of the patient the learned patient data.

17. The system of claim 14, in which the patient data is originated from a medical device that is monitoring the patient.

18. The system of claim 1, in which the processor is further configured to:
cause the first message to be transmitted to a first one of the network destinations, receive an acknowledgement from an operator at the first network destination about a readiness to treat the patient by the person or facility of the first network destination, and
record a readiness time difference from when the prepared first draft message was caused to be transmitted, to when the acknowledgement was received.

19. A system for automatically launching electronic messages for treating patients in a center having at least two distinct network destinations, each destination associated with persons or facilities to treat the patients, the system comprising:
at least one means for storing:
a plurality of possible characterizations of ailments,
a plurality of message templates, and
association data that associates at least some of the possible characterizations with at least some of the message templates;
means for receiving a learned characterization of an ailment of an incoming patient;
means for transmitting an inquiry to one or more of at least one of a medical provider or a facility to confirm availability, the means for transmitting the inquiry including a request to the at least one of the medical provider or facility to send a response to confirm availability;
one or more means for:
generating at least a first and a second draft message,
receiving the learned characterization,
determining whether the received characterization matches one of the possible characterizations,
automatically selecting at least a first message template and a second message template, the first message template different from the second message template, from the plurality of message templates associated with the learned characterization that matches one of the possible characterizations;
applying the first message template to the first draft message to generate a first message, the first message including the inquiry to the at least one of the medical provider or the facility to send the response to confirm availability,
applying the second message template to the second draft message to generate a second message, the second message including the inquiry to the at least one of the medical provider or the facility to send the response to confirm availability,
causing the system to automatically prepare for transmission the first message to a first network destination and the second message to a second network destination, the first network destination distinct from the second network destination,
selecting, based on the first message template, between causing the first message-to be transmitted automatically or requiring a user to perform a separate action through an operator module to allow the first message to be transmitted, and
receiving the response to confirm availability from the first network destination and the second network destination.

20. A messaging system for automatic message transmission within a patient treatment center having at least two distinct network destinations, each network destination associated with at least one of a person or an organizational unit of the patient treatment center, the messaging system comprising:
a memory configured to store:
a plurality of ailment characterizations,
a plurality of message templates, and
association data that associates one or more ailment characterizations with one or more message templates;
an operator module configured to receive a learned characterization of a patient ailment;
one or more processors configured to:
generate a first and a second draft message,
receive the learned characterization,
determine one or more ailment characterizations based on the learned characterization,
automatically select at least two different message templates, from the plurality of message templates, associated with the one or more determined ailment characterizations;
applying one of the at least two different message templates to the first draft message to generate a first message and apply the other of the at least two different message templates to the second draft message to generate a second message,
automatically prepare the first and second messages to the at least two distinct network destinations, the first message to cause at least one of the two distinct network destinations to prepare for an interaction with the patient upon receipt of the first message, the first and second messages including an inquiry to confirm availability of the at least one of the person or the organizational unit;
select, based on the at least two different message templates, between causing at least one of the first message or the second message to be transmitted automatically or requiring a user to perform a separate action through the operator module to allow the at least one of the first message or the second message to be transmitted; and
receive confirmation from the at least one of the two distinct network destinations, the confirmation indicating the availability of the least one of the two distinct network destinations to treat the patient.
